(19) United States
(12) Patent Application Publication
(10) Pub. No.: US 2026/0348934 A1
(43) Pub. Date: Nov. 5, 2026

(54) ANALYSIS DEVICE, ANALYSIS METHOD, AND PROGRAM

(71) Applicant: HORIBA, LTD., Kyoto-shi, Kyoto (JP)

(72) Inventors: Kyoji SHIBUYA, Kyoto-shi, Kyoto (JP); Kodai NIINA, Kyoto-shi, Kyoto (JP); Naoki OKUDA, Kyoto-shi, Kyoto (JP); Daisuke MIYAWAKI, Kyoto-shi, Kyoto (JP); Takuya IDO, Kyoto-shi, Kyoto (JP)

(73) Assignee: HORIBA, LTD., Kyoto-shi, Kyoto (JP)

(21) Appl. No.: 19/487,261

(22) PCT Filed: May 16, 2024
(86) PCT No.: PCT/JP2024/018060

§ 371 Date: Nov. 24, 2025

(30) Foreign Application Priority Data
Jun. 1, 2023 (JP) 2023-090770

(51) Int. Cl. G01N 33/00; G01N 21/39

(52) U.S. Cl. CPC G01N 33/007; G01N 21/39; G01N 2021/399

(57) ABSTRACT

To easily determine whether or not an obtained analysis result is abnormal, an analysis device includes an irradiation unit, a detection unit, and an arithmetic unit. The irradiation unit includes a laser that emits measurement light toward a sample gas. The detection unit detects the measurement light after passing through the sample gas. The arithmetic unit outputs a signal for determining whether or not the analysis result of the predetermined gas is abnormal, based on a residue between detection signal information based on a detection signal obtained when the detection unit detects the measurement light, and reference signal information based on a reference signal obtained in advance by causing the predetermined gas to absorb the measurement light.

### TECHNICAL FIELD

The present invention relates to an analysis device and an analysis method of analyzing a predetermined gas contained in a sample gas, and a program causing a computer to perform the analysis method.

### BACKGROUND ART

Conventionally, a device is known that emits measurement light toward a sample gas, and analyzes a predetermined gas contained in the sample gas, based on absorption spectrum obtained from the measurement light after passing through the sample gas. In this device, the analysis of the predetermined gas is performed based on characteristics indicating that wavelengths of absorbed light are different for individual gases, and that absorbed light amount depends on gas concentration. Therefore, if the obtained absorption spectrum is different from expected one, it can be determined that an analysis result is abnormal.

For instance, in a gas concentration measurement device that uses a TDLAS (Tunable Diode Laser Absorption Spectroscopy, or wavelength tunable semiconductor laser absorption spectral) measurement method, it is known to determine whether or not it is the state where an analysis result of a gas to be measured cannot be obtained, based on parameters that can affect on the absorption spectrum (such as total pressure and temperature of the sample gas), a shape of the absorption spectrum, a peak position, and the like (see Patent Citation 1, for example).

### PRIOR ART CITATIONS

### Patent Citation

Patent Citation 1: JP-A-2011-242222

### SUMMARY OF INVENTION

### Technical Problem

As described above, in order to determine whether or not it is the state where a normal analysis result cannot be obtained based on parameters such as total pressure and temperature of the sample gas, a shape of the absorption spectrum, and a peak position, it is necessary to determine, for example, a degree of difference of various parameters, a shape of the spectrum, and/or a peak position for determining abnormality, based on experience. In other words, in a conventional abnormality determination method, it is necessary to determine based on proficient experience or the like, and it may not be easy to determine whether or not it is the state where a normal analysis result cannot be obtained. For instance, a user with little experience may overlook various parameters, an absorption spectrum shape, and/or a peak position, which are abnormal, and may wrongly determine that the analysis result obtained from the absorption spectrum or the like including abnormality is a normal result.

It is an object of the present invention to easily determine whether or not the obtained analysis result is abnormal, without requirement of proficient experience.

### Technical Solution

Hereinafter, a plurality of embodiments are described as means for solving the problem. These embodiments can be arbitrarily combined as necessary.

An analysis device according to an aspect of the present invention is an analysis device that analyzes a predetermined gas contained in a sample gas. The analysis device includes an irradiation unit, a detection unit, and an arithmetic unit. The irradiation unit includes a laser that emits measurement light toward the sample gas. The detection unit detects the measurement light after passing through the sample gas. The arithmetic unit analyzes the predetermined gas based on a detection signal obtained when the detection unit detects the measurement light. In addition, the arithmetic unit outputs a signal for determining whether or not the analysis result of the predetermined gas is abnormal, based on a residue between detection signal information based on the detection signal and reference signal information based on a reference signal indicating absorption of the measurement light by the predetermined gas.

In the analysis device described above, the signal for determining whether or not the analysis result of the predetermined gas is abnormal is output, based on a residue between detection signal information based on an actual detection signal obtained by causing the measurement light to pass through the sample gas, and reference signal information based on a reference signal indicating absorption of the measurement light by the predetermined gas. The residue between the detection signal information and the reference signal information indicates an amount of shift between the detection signal that is actually detected and the reference signal, and is calculated by predetermined information processing using the detection signal information and the reference signal information. For this reason, in the analysis device described above, it can be easily determined whether or not the analysis result of the predetermined gas is abnormal, without using a method requiring proficient experience.

In the analysis device described above, the arithmetic unit may output a signal indicating that an analysis result of the predetermined gas is abnormal if the residue is a predetermined threshold value or more. In this way, if the detection signal actually obtained is largely dissociated from the reference signal obtained in advance, it can be appropriately determined that the analysis result of the predetermined gas is abnormal.

In the analysis device described above, the arithmetic unit may output a signal for indicating that the sample gas contains unknown gas other than the predetermined gas, if the residue is a predetermined threshold value or more. In this way, it is possible to know that the analysis result of the predetermined gas is abnormal because the unknown gas is contained.

In the analysis device described above, the arithmetic unit may calculate the residue by regression analysis using the detection signal information and the reference signal information. In this way, a shift between the detection signal information and the reference signal information can be easily calculated.

In the analysis device described above, the detection signal information may include detection feature quantity that indicates features of the detection signal. In addition, the reference signal information may include reference feature quantity that indicates features of the reference signal. In this way, it is possible to reduce the number of parameters that are used in calculation for calculating the residue.

In the analysis device described above, the detection signal information may be a detection absorption spectrum generated based on the detection signal. The reference signal information may be a reference absorption spectrum generated based on the reference signal. In this way, the residue can be calculated based on comparison between the detection absorption spectrum and the reference absorption spectrum, for example, and it can be determined whether or not the analysis result is abnormal.

In the analysis device described above, the arithmetic unit may output a signal for determining whether or not the analysis result of the predetermined gas is abnormal, based on not only the residue but also at least one of sample gas pressure, temperature of the analysis device, a peak position of a spectrum generated from the detection signal, and a spread width of the spectrum, when the detection signal is obtained. In this way, it is possible to identify abnormality caused by sample gas pressure, temperature of the analysis device, or a peak position of the spectrum generated from the detection signal.

An analysis method according to another aspect of the present invention is an analysis method of analyzing a predetermined gas contained in a sample gas. The analysis method includes:
causing a laser to emit measurement light toward a sample gas;
detecting the measurement light after passing through the sample gas;
analyzing the predetermined gas based on a detection signal obtained by detecting the measurement light; and
outputting a signal for determining whether or not the analysis result of the predetermined gas is abnormal, based on a residue between detection signal information based on the detection signal, and reference signal information based on a reference signal indicating absorption of the measurement light by the predetermined gas.

In the analysis method described above, the signal for determining whether or not the analysis result of the predetermined gas is abnormal is output, based on the residue between the detection signal information based on an actual detection signal obtained by causing the measurement light to pass through the sample gas, and the reference signal information based on a reference signal indicating absorption of the measurement light by the predetermined gas. The residue between the detection signal information and the reference signal information indicates an amount of shift between the detection signal that is actually detected and the reference signal, and can be calculated by predetermined information processing using the detection signal information and the reference signal information. For this reason, in the analysis method described above, it can be easily determined whether or not the analysis result of the predetermined gas is abnormal, without using a method requiring proficient experience.

A program according to still another aspect of the present invention is a program that causes a computer to perform the analysis method described above.

### Advantageous Effects

It is possible to easily and automatically determine whether or not an analysis result of a predetermined gas contained in a sample gas is abnormal, without using a method requiring proficient experience.

### BRIEF DESCRIPTION OF DRAWINGS

FIG. 1 is a diagram illustrating a configuration of an analysis device.

FIG. 2 is a flowchart illustrating an analysis operation in the analysis device.

FIG. 3 is a flowchart illustrating an example of a specific analysis operation of an analysis target gas.

FIG. 4 is a flowchart illustrating an example of a specific operation for outputting a signal for determining whether or not the analysis result of the analysis target gas is abnormal.

FIG. 5 is a diagram illustrating meaning of a residue.

### DESCRIPTION OF EMBODIMENTS

### 1. First Embodiment

### (1) Configuration of Analysis Device

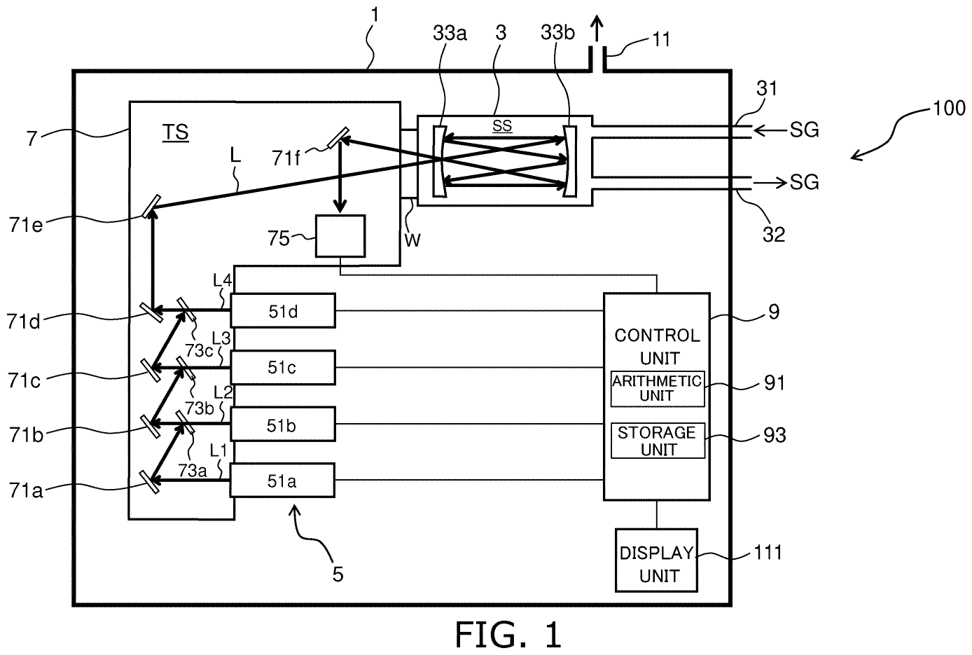

Hereinafter, with reference to FIG. 1, an analysis device 100 according to a first embodiment is described. FIG. 1 is a diagram illustrating a configuration of the analysis device. The analysis device 100 is a device that analyzes a predetermined gas contained in a sample gas SG such as a gas generated from a chemical plant, an exhaust gas from an automobile, or an exhaust gas generated from a flue, for example.

A gas that can be analyzed by the analysis device 100 (referred to as an analysis target gas) is, for example, carbon dioxide (CO₂), carbon monoxide (CO), sulfur oxide (SOx) (such as sulfur dioxide (SO₂)), ammonia (NH₃), nitrogen oxide (NOX) (such as nitric monoxide (NO), nitric dioxide (NO₂), or dinitrous monoxide (N₂O)), hydrogen chloride (HCl), water (H₂O), various hydrocarbons (such as methane (CH₄), ethane (C₂H₆), acethylene (C₂H₂), propane (C₃H₈), ethylene (C₂H₄), hexane (n-C₆H₁₄), propylene (C₃H₆), isobutylene (i-C₄H₈), butane (n-C₄H₁₀), cyclohexane (C₆H₁₂), butadiene (C₄H₆), isobutane (i-C₄H₁₀), isopentane (i-C₅H₁₂), benzene (C₆H₆), toluene (C₆H₅CH₃), xylene (C₈H₁₀), and methylcyclohexane (C₇H₁₄)), hydrogen sulfide (H₂S), methanol (CH₃OH), phosgene (COCl₂), chloroethylene (C₂H₃Cl), methyl nitrite (CH₃ONO), hydrogen (H₂), oxygen (O₂), hydrogen fluoride (HF), trifluoropropene (C₃H₃F₃), or the like. The analysis device 100 can analyze a mixed gas containing a plurality of gases described above, as the analysis target gas.

In addition, the sample gas SG may contain a gas described above and/or other gas as a gas that affects an analysis result of the analysis target gas (referred to as an interference gas). The interference gas may have absorption spectrum that overlaps a part of absorption spectrum of the analysis target gas. If the absorption spectrum of the interference gas and the absorption spectrum of the analysis target gas overlap partially, an analysis result of the analysis target gas is affected.

In addition, in the analysis device 100 that uses laser light as measurement light, the absorption spectrum is affected not only by the interference gas, but also by concentration change of a gas component (referred to as an accompanying component) that accompanies in high concentration (e.g., approximately a few percent to a few tens percent). Specifically, due to the concentration change of the accompanying component, a shape of the absorption spectrum is changed.

As illustrated in FIG. 1, the analysis device 100 includes a casing 1, a filling unit 3, an irradiation unit 5, a propagation unit 7, and a control unit 9.

The casing 1 constitutes a main body of the analysis device 100, and houses the filling unit 3, the irradiation unit 5, and the propagation unit 7 in its inside space. The casing 1 may be an inner pressure explosion-proof container, or may not support the explosion-proof function. In order to constitute the casing 1 as an explosion-proof container, the inside space of the casing 1 is filled with a gas for preventing explosion, with higher pressure than outside of the casing 1. The gas for preventing explosion is a gas that does not contain a flammable gas, and is air for example. By constituting the casing 1 as the inner pressure explosion-proof container, the inside space of the casing 1 can be safely prevented from explosion. The casing 1 is provided with a discharge port 11 disposed near the filling unit 3. The gas for preventing explosion introduced to the inside space of the casing 1 is discharged from the discharge port 11.

The filling unit 3 is a member having a sampling space SS. The sampling space SS of the filling unit 3 can be filled with the sample gas SG. In order to fill the sample gas SG into the sampling space SS, the filling unit 3 is provided with an inlet 31 for introducing the sample gas SG into the sampling space SS, and an outlet 32 for discharging the sample gas SG in the sampling space SS. During analysis of the analysis target gas contained in the sample gas SG, the sample gas SG is filled through the inlet 31 into the sampling space SS, and then is discharged through the outlet 32, and this flow is continued.

The sampling space SS has lower pressure than the inside space of the casing 1 and a propagation space TS of the propagation unit 7, including the period while the sample gas SG is being filled. In this way, the sample gas SG filled in the sampling space SS is prevented from leaking into the inside space of the casing 1 or the propagation space TS of the propagation unit 7, and explosion-proof measurement for the analysis device 100 can be appropriately performed.

In addition, the sampling space SS is provided with a first reflection member 33a and a second reflection member 33b. The first reflection member 33a and the second reflection member 33b cause measurement light L that enters the sampling space SS to be multiply reflected, and then to propagate to the propagation space TS of the propagation unit 7 (described later). In this way, it is possible to increase a light path length of the measurement light L that passes through the sample gas SG filled in the sampling space SS.

The first reflection member 33a is disposed at a position close the propagation unit 7 in the sampling space SS. The first reflection member 33a reflects the measurement light L and causes the measurement light L to propagate the second reflection member 33b. In addition, it causes the measurement light L after the multiple reflection to propagate to the propagation unit 7. Therefore, for example, the first reflection member 33a is a member (such as a mirror) that can reflect the measurement light L and has a passing through hole for the measurement light L formed at a predetermined position. In this first reflection member 33a, the measurement light L that has propagated from the propagation unit 7 passes through the passing through hole and enters into the sampling space SS. The measurement light L that has entered the sampling space SS is reflected multiply between the first reflection member 33a and the second reflection member 33b, and then returns to the propagation unit 7 through the passing through hole of the first reflection member 33a.

The second reflection member 33b is disposed at a position apart from the propagation unit 7 in the sampling space SS. The second reflection member 33b reflects the measurement light L to the first reflection member 33a. The second reflection member 33b is a member such as a mirror that reflects the measurement light L.

Note that configuration of the first reflection member 33a and/or the second reflection member 33b in the filling unit 3 is not limited to that described above. For instance, it may be possible not to form the passing through hole for the measurement light L in the first reflection member 33a. In this case, for example, the measurement light L can enter and exit through a portion that is not blocked by the first reflection member 33a of the filling unit 3. In addition, the passing through hole described above may not be common for the measurement light L entering the sampling space SS and the measurement light L going out from the sampling space SS. In other words, it may be possible to separately dispose a passing through hole that is configured to cause the measurement light L to enter the sampling space SS, and a passing through hole that is configured to cause the measurement light L to go out from the sampling space SS.

The irradiation unit 5 generates the measurement light L. The measurement light L generated from the irradiation unit 5 is guided by the propagation unit 7 to the filling unit 3. The irradiation unit 5 includes a plurality of light sources 51a to 51d. The plurality of light sources 51a to 51d output a plurality of element lights L1 to L4 having different wavelength regions. The plurality of light sources 51a to 51d are each a quantum cascade laser (QCL), for example, which is one type of a semiconductor laser. The light sources 51a to 51d each have an oscillation wavelength that can be modulated with current or voltage. Note that other type of laser may be used for the light sources 51a to 51d as long as its oscillation wavelength is variable. For instance, it may be a laser whose oscillation wavelength can be modulated by changing temperature or the like.

The element lights L1 to L4 generated from the plurality of light sources 51a to 51d are multiplexed in the propagation space TS of the propagation unit 7, to be the measurement light L that propagates to the filling unit 3. In other words, the measurement light L includes the plurality of element lights L1 to L4 having different wavelength regions. Because the measurement light L includes the plurality of element lights L1 to L4, it is possible, for example, to analyze a plurality of analysis target gases and/or interference gas, which have absorption peaks for wavelength regions of the element lights L1 to L4.

Note that an arbitrary number of light sources can be disposed in the irradiation unit 5. For instance, only one light source is disposed in the irradiation unit 5. In other words, the number of the element light may be one.

The propagation unit 7 is disposed between the filling unit 3 and the irradiation unit 5. Specifically, as illustrated in FIG. 1, the propagation unit 7 is fixed in a state where the plurality of light sources 51a to 51d are each partly inserted in a portion of the propagation unit 7 corresponding to one end thereof. On the other hand, the filling unit 3 is fixed to a portion of the propagation unit 7 corresponding to the other end thereof, via an optical window W.

The propagation unit 7 has the propagation space TS. In order to realize an explosion-proof state of the propagation space TS of the propagation unit 7, it may be possible to constitute the propagation unit 7 as an inner pressure explosion-proof container. Alternatively, if it is not necessary to realize an explosion-proof state of the propagation space TS, the propagation unit 7 may not be the inner pressure explosion-proof container. The propagation space TS of the propagation unit 7 can be formed, for example, by hollowing out a metal block to form a space, and closing the space in the metal block with a lid member. The metal members (the metal block and the lid member) that form the propagation unit 7 are, for example, aluminum members. If the propagation unit 7 is formed by aluminum members, the aluminum members may be black anodized to have black color for countermeasures against stray light. Also in case where the propagation unit 7 is formed by other material, it is preferred that the propagation unit 7 should have black color for countermeasures against stray light. With the configuration described above, the robust propagation unit 7 can be easily formed.

A plurality of mirrors are disposed in the propagation space TS of the propagation unit 7. If the irradiation unit 5 includes a plurality of light sources, the element lights L1 to L4 output from the irradiation unit 5 propagate to the filling unit 3 while propagation paths thereof are changed by the mirrors. In other words, the measurement light L propagates in the propagation space TS while being reflected by at least one mirror. Specifically, a first mirror 71a, a second mirror 71b, a third mirror 71c, a fourth mirror 71d, and a fifth mirror 71e are disposed in the propagation space TS. In addition, a first optical element 73a, a second optical element 73b, and a third optical element 73c are disposed in the propagation space TS.

Note that the number of mirrors disposed in the propagation unit 7 are arbitrary, in accordance with the number of the light sources disposed in the irradiation unit 5, setting of light path of the measurement light L in the propagation space TS, or the like, for example.

The first mirror 71a reflects the element light L1 to the first optical element 73a. The first optical element 73a reflects the element light L1, while it transmits the element light L2. In other words, the first optical element 73a multiplexes the element light L1 and the element light L2. The element light L1 and the element light L2 multiplexed by the first optical element 73a propagate along the same light path to the second mirror 71b.

The second mirror 71b reflects the element light L1 and the element light L2 multiplexed by the first optical element 73a to the second optical element 73b. The second optical element 73b reflects the multiplexed element light L1 and element light L2, while it transmits the element light L3. In other words, the second optical element 73b multiplexes the element light L1, the element light L2, and the element light L3. The element lights L1 to L3 multiplexed by the second optical element 73b propagate along the same light path to the third mirror 71c.

The third mirror 71c reflects the element lights L1 to L3 multiplexed by the second optical element 73b to the third optical element 73c. The third optical element 73c reflects the multiplexed element lights L1 to L3, while it transmits the element light L4. In other words, the third optical element 73c multiplexes the element light L1, the element light L2, the element light L3, and the element light L4. The element lights L1 to L4 multiplexed by the third optical element 73c propagate along the same light path to the fourth mirror 71d.

The fourth mirror 71d reflects the element lights L1 to L4 multiplexed by the third optical element 73c to the fifth mirror 71e. The element lights L1 to L4 multiplexed as described above become the measurement light L.

The fifth mirror 71e changes the propagation path of the measurement light L including the multiplexed element lights L1 to L4 to the direction of the filling unit 3. The measurement light L on the propagation path changed by the fifth mirror 71e passes through the optical window W to propagate into the sampling space SS of the filling unit 3. These mirrors are disposed in the propagation space TS, and hence it is possible to maintain clean state of the mirrors by preventing unnecessary gas from entering from the outside.

In addition, a detection unit 75 is disposed in the propagation space TS of the propagation unit 7. The detection unit 75 detects the measurement light L that has entered the sampling space SS and has been multiply reflected. The detection unit 75 is a quantum photoelectric device, for example. Other than that, a semiconductor detection element, a thermal photodetection element such as a thermopile, or the like can be used as the detection unit 75. As illustrated in FIG. 1, the propagation space TS is provided with a sixth mirror 71f that changes the propagation path of the measurement light L, which has been multiply reflected in the sampling space SS and has returned to the propagation unit 7, to the direction of the detection unit 75.

The inside of the propagation space TS of the propagation unit 7 may be purged using a purge gas. The purge gas is a gas that does not react with the measurement light L (e.g., does not absorb the measurement light L), and is nitrogen gas or an inert gas, for example. In this way, the purge gas flows in the propagation space TS, and hence the first mirror 71a to the sixth mirror 71f and the detection unit 75 disposed in the propagation space TS can be in clean state. In addition, for example, in the propagation space TS, the measurement light L (the element lights) is prevented from being absorbed by a component in the propagation space TS.

The light path of the measurement light L from the propagation unit 7 to the filling unit 3 is determined by the first mirror 71a to the sixth mirror 71f. In order to cause the measurement light L to appropriately propagate to the filling unit 3, inclination angles of these mirrors are adjustable. For instance, by rotating screws (not shown) disposed in the propagation space TS, the inclination angles of the first mirror 71a to the fourth mirror 71d can be adjusted.

The control unit 9 is a computer system that includes a CPU, a storage device (such as a RAM or a ROM), and various interfaces. The control unit 9 may be a system including the above devices separately, or may be a system on chip (SoC) including the above devices integrated on a single chip. The control unit 9 controls component elements of the analysis device 100.

The control unit 9 includes an arithmetic unit 91 and a storage unit 93. The arithmetic unit 91 is the CPU and the various interfaces of the computer system constituting the control unit 9, and performs various information processing about control of the analysis device 100, analysis of the analysis target gas, and the like. It may also be possible that a part or the whole of the information processing performed by the arithmetic unit 91 is realized by executing a program stored in the storage unit 93. In addition, a part of the information processing described above may be realized by hardware.

The arithmetic unit 91 inputs a signal about the measurement light L (referred to as a detection signal), which has passed through the sample gas SG filled in the sampling space SS of the filling unit 3 and has been detected by the detection unit 75, from the detection unit 75, and analyzes the analysis target gas contained in the sample gas SG based on the detection signal input from the detection unit 75.

In addition, the arithmetic unit 91 outputs a signal for determining whether or not the analysis result of the predetermined gas is abnormal, based on a residue between detection signal information based on the detection signal described above, and reference signal information based on a reference signal indicating absorption of the measurement light by the predetermined gas. Specifically, if the residue between the detection signal information and the reference signal information is a predetermined threshold value or more, the arithmetic unit 91 determines that there is a large shift between the detection signal information and the reference signal information, and outputs a signal for indicating that an analysis result of the analysis target gas is abnormal.

The storage unit 93 stores a program that is executed by the arithmetic unit 91, and various parameters related to control of the analysis device 100, analysis of the analysis target gas, and the like. The storage unit 93 stores the reference feature quantity described above.

The control unit 9 is connected to a display unit 111. The display unit 111 displays information about the analysis device 100, such as an analysis result of the analysis target gas by the analysis device 100. For instance, the display unit 111 is a thin display such as a liquid crystal display or an organic EL display. In addition, the display unit 111 may have information input means such as a touch panel.

### (2) Operation of Analysis Device

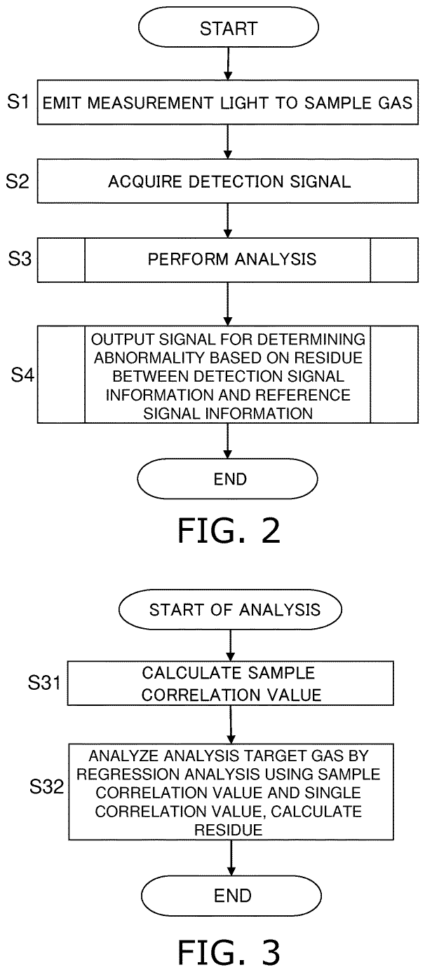

Next, with reference to FIG. 2, gas analysis operation using the analysis device 100 is described. FIG. 2 is a flowchart illustrating the analysis operation in the analysis device.

First, in the state where supply of the sample gas SG to the sampling space SS is maintained, the arithmetic unit 91 controls the light sources 51a to 51d, to emit the measurement light L to the sampling space SS in which the sample gas SG is filled (Step S1).

Next, the arithmetic unit 91 receives the detection signal related to the measurement light L after passing through the sampling space SS from the detection unit 75 (Step S2). After receiving the detection signal, the arithmetic unit 91 detects the measurement light L with the detection unit 75, to analyze the analysis target gas based on the obtained detection signal (Step S3).

In addition, the arithmetic unit 91 outputs a signal for determining whether or not an analysis result of the analysis target gas contained in the sample gas SG is abnormal, based on the residue between the detection signal information and the reference signal information (Step S4). The detection signal information is information based on the detection signal obtained in Step S2. The reference signal information is information based on the reference signal that indicates absorption of the measurement light L by the analysis target gas.

### (3) Specific Example of Analysis Operation of Analysis Target Gas

Hereinafter, described is an example of the analysis of the analysis target gas performed in Step S3 described above. In this example, the arithmetic unit 91 calculates concentration of the analysis target gas contained in the sample gas SG, using the detection signal information that is information based on the detection signal, and the reference signal information that is information based on the reference signal that indicates absorption of the measurement light L by the analysis target gas.

In this example, a plurality of feature quantities extracted from the detection signal (referred to as detection feature quantities) are the detection signal information. In addition, a plurality of corresponding feature quantities extracted from the reference signal (referred to as reference feature quantities) are reference signal information. In this way, compared with the case where the detection signal and the reference signal themselves are used, the number of data sets can be reduced, and hence a load to calculate concentration of the analysis target gas can be reduced. The reference feature quantities are extracted in advance and are stored in the storage unit 93.

Note that the reference signal may be one obtained by measuring intensity of the measurement light L absorbed by the analysis target gas using the detection unit 75 or the like, or may be one obtained from a database that stores absorption spectra of various gases (absorption peak positions (wavelengths), absorption peak intensities).

Hereinafter, an example of a method of extracting the reference feature quantity is described. In the following description, an example where a “single correlation value” is extracted as the reference feature quantity is described. The single correlation value is a correlation value per unit concentration of each of a measurement target component (the analysis target gas) and interference components (interference gases), determined from an intensity related signal (the reference signal) and a plurality of feature signals of the measurement target component and the interference components, which separately exist.

The extraction of the single correlation value can be performed by the following procedure. First, a signal output from the detection unit 75, when the measurement light L is absorbed by only the analysis target gas, is acquired as the intensity related signal (i.e., the reference signal). In addition, a signal obtained when the measurement light L is absorbed by only the interference gas to be considered in the analysis is also acquired as the intensity related signal.

The acquisition of the intensity related signal is realized, for example, by introducing a gas containing the measurement target component or each interference component at given concentration into the sampling space SS of the filling unit 3, detecting intensity of the measurement light L after passing through the sampling space SS filled with the gas using the detection unit 75, and taking the logarithm of the signal input from the detection unit 75 (i.e., the signal indicating intensity of the measurement light L) to be the intensity related signal. Other than that, it may also be possible to select the absorption spectrum of the measurement target component or each interference component, from the database that stores the absorption spectra (the absorption peak positions (wavelengths), absorption peak intensities) of various gases, and to acquire the intensity related signal based on the peak position of the selected absorption spectrum and the peak intensity at the position.

In order to acquire the intensity related signal, the wavelength of the measurement light L is scanned by controlling the light sources 51a to 51d. In other words, the intensity related signal is acquired as data in which time from start of scanning is associated with a signal value acquired when the measurement light L having a wavelength determined at the time is absorbed by the gas. The same is also true for the detection signal.

Next, the single correlation value is extracted from the intensity related signal. As to the single correlation value, for example, based on the acquired intensity related signal and a feature signal Fᵢ(t) for extracting the correlation value of the signal, a plurality of the single correlation values can be extracted using the following equation (Equation 1).

$${S}_{i} = {\int }_{{t}_{1}}^{{t}_{2}} L ( t ) \cdot {F}_{i} ( t ) dt ( i = 1 , 2 , 3 , ⋯ , n ) \qquad [ Equation 1 ]$$

When extracting the single correlation value using the above Equation 1, L(t) is the intensity related signal. Sᵢ in Equation 1 is the correlation value for calculating the single correlation value. Further, t₁ and t₂ define limits of integration in Equation 1. The limit t₁ can be 0, for example. The limit t₂ can be T, for example (T is a scan period of the wavelength of the measurement light L from the light sources 51a to 51d). Further, n is the number of the single correlation values to be extracted. The number n is preferably more than the sum of the number of the measurement target components and the number of interference components to be considered.

In the above Equation 1, the feature signal Fᵢ(t) is a function that can show a feature of the waveform of (logarithm of) the intensity L(t) more than a sine function. Specifically, the feature signal Fᵢ(t) can be, for example, a function that expresses a shape close to the spectrum, such as a sine function, a function based on Lorenz function, a partial differentiation function of the function based on the Lorenz function with respect to Lorenz width, a function based on a Voigt function, or a function based on a Gauss function. Note that n feature signals Fᵢ(t) are preferably functions that are perpendicular to each other (i.e., functions having no correlation).

After extracting the correlation values Sᵢ using Equation 1, the single correlation value is calculated from the extracted correlation values Sᵢ. Specifically, using Equation 1, each of the extracted correlation values is divided by concentration of the analysis target component or each interference component, to calculate the correlation values sᵢ (i=1, 2, 3, . . . n) per unit concentration as the single correlation value. The extracted single correlation value is stored in the storage unit 93.

Note that in the case where the single correlation value is calculated as described above, the correlation value Sᵢ extracted using Equation 1 minus a reference correlation value may be used for calculation of the single correlation value. The reference correlation value is a correlation value extracted from a signal acquired by detecting the measurement light L after passing through the space without the analysis target component or the interference component (i.e., a background signal), using Equation 1. For instance, the reference correlation value is calculated in advance using a signal acquired when a gas that does not affect the measurement light L (a zero gas) is filled in the sampling space SS, and Equation 1 described above, and is stored in the storage unit 93.

Hereinafter, with reference to FIG. 3, an example of the analysis operation of the analysis target gas will be described more specifically. FIG. 3 is a flowchart illustrating an example of a specific analysis operation of the analysis target gas. First, based on a detection signal received from the detection unit 75, the arithmetic unit 91 calculates a sample correlation value that is the correlation value about the detection signal (Step S31).

Specifically, in Equation 1 described above for calculating the feature quantity, the arithmetic unit 91 calculates the sample correlation values S_{d1}, S_{d2}, S_{d3}, . . . S_{dn}, using L(t) that is a logarithm signal of the detection signal acquired in Step S2, and Fᵢ(t) that is the feature signal used in extraction of the single correlation value. Note that also in calculation of the sample correlation value, the sample correlation value may be calculated by subtracting the reference correlation value described above from the correlation value calculated using Equation 1.

After calculating the sample correlation value, the arithmetic unit 91 calculates concentrations of the measurement target component and/or the interference component, by regression analysis of the above sample correlation value and the corresponding single correlation value, and calculates the residue between the sample correlation value and the corresponding single correlation value (Step S32). Specifically, the arithmetic unit 91 calculates concentrations of the analysis target gas and/or the interference gas by solving the simultaneous equations (Equation 2), which include a plurality of equations expressing relationships among the sample correlation value, the corresponding single correlation value, and the concentrations of the analysis target component and/or the interference component to be considered.

S_{d1}=s₁₁*C1+s₁₂*C2+s₁₃*C3+ . . . +s₁ₘ*Cm

S_{d2}=s₂₁*C1+s₂₂*C2+s₂₃*C3+ . . . +s₂ₘ*Cm

S_{d3}=s₃₁*C1+s₃₂*C2+s₃₃*C3+ . . . +s₃ₘ*Cm

. . .

S_{dn}=sₙ₁*C1+sₙ₂*C2+sₙ₃*C3+ . . . +sₙₘ*Cm [Equation 2]

In Equation 2, m is the sum of the number of analysis target components and the interference components to be considered (i.e., the interference components predicted to be contained in the sample gas SG) (preferably m<n holds). S_{d1}, S_{d2}, S_{d3}, . . . S_{dn} are sample correlation values calculated in Step S3. Further, sᵢₖ (i=1, 2, . . . n, k=1, 2, . . . m) are the single correlation values corresponding to S_{di} for a specific analysis target component or interference component having an identification symbol k. Ck is concentration of a specific component (analysis target component/interference component) having the identification symbol k.

The arithmetic unit 91 solves the simultaneous equations expressed in Equation 2, to calculate specific values of C1, C2, C3, . . . Cm. The simultaneous equations of Equation 2 in which m<n holds can be solved using a least-square method. Specifically, the arithmetic unit 91 calculates values of C1, C2, C3, . . . Cm, in which the sum of squares of the difference between the left side (i.e., S_{di}) and the right side (i.e., the expression of the products of sᵢₖ and Ck) in Equation 2 becomes minimum. The sum of squares of the differences between the left side and the right side in Equation 2 is the residue indicating the shift between S_{di} (the sample correlation value) and sᵢₖ (the single correlation value). In other words, the arithmetic unit 91 calculates values of C1, C2, C3, . . . Cm in which the residue becomes minimum, by the least-square method.

Other than that, the arithmetic unit 91 can also calculate concentrations of the analysis target gas and/or the interference gas, by curve fitting between the above sample correlation values and the corresponding single correlation values. Specifically, with respect to coordinate points (Sᵢ₁, Sᵢ₂, . . . , sᵢₘ, S_{di}) (i=1, 2, . . . n) on the m+1 dimensional coordinate space in which n sample correlation values and the single correlation values are coordinated, using a curve on the m+1 dimensional coordinate space expressed by S=s₁*C1+s₂*C2+ . . . sₘ*Cm (S is the sample correlation value, s₁, s₂, . . . sₘ are the single correlation values, C1, C2, . . . Cm are the concentrations of the measurement target components/the interference components), C1, C2, . . . Cm (i.e., the concentrations of the measurement target components/the interference components) can be calculated by curve fitting, using the least-square method with fitting parameters C1, C2, . . . Cm.

In the curve fitting described above, C1, C2, . . . Cm are determined as specific values (concentrations), and hence the curve expressed by S=s₁*C1+s₂*C2+ . . . sₘ*Cm is the equation of the relationship between the sample correlation values (S) and the single correlation values (s₁, s₂, . . . sₘ). Further, the shift between the curve expressed by S=s₁*C1+s₂*C2+ . . . sₘ*Cm and coordinate points of the sample correlation values and the single correlation values (sᵢ₁, sᵢ₂, . . . , sᵢₘ, S_{di}) (e.g., the sum of squares of distances between the above curve and individual coordinate points) is calculated as the residue.

Note that the curve fitting described above may be performed in consideration of influences of a wavelength shift in the absorption spectrum, spreading of the absorption spectrum, and the like.

### (4) Specific Example of Output Operation of Signal for Determining Whether or not Analysis Result is Abnormal

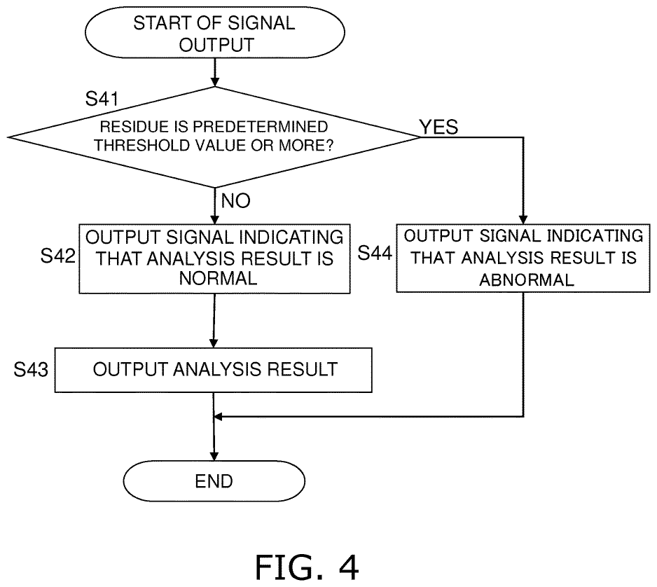

Hereinafter, with reference to FIG. 4, described is an example of a specific operation of outputting a signal for determining whether or not an analysis result of the analysis target gas is abnormal, which is performed in Step S4 described above. FIG. 4 is a flowchart illustrating an example of a specific operation of outputting a signal for determining whether or not an analysis result of the analysis target gas is abnormal.

In the signal output operation described above, the arithmetic unit 91 first determines whether or not the residue, which was calculated in the analysis of the analysis target gas described above, is a predetermined threshold value or more (Step S41). This predetermined threshold value is determined based on a criterion such as a degree of the dissociation between the sample correlation value and the single correlation value, which is regarded as abnormal, for example, and is stored in the storage unit 93.

If the residue is less than the predetermined threshold value (“No” in Step S41), the arithmetic unit 91 outputs a signal for indicating that the analysis result is normal (Step S42). For instance, the arithmetic unit 91 outputs the signal for displaying that the analysis result is normal to the display unit 111, outputs a signal producing sound for informing that the analysis result is normal to a predetermined device (such as an alarm device), and/or outputs a signal for outputting that the analysis result is normal to a predetermined device (such as a printer). In addition, the arithmetic unit 91 may output the signal for outputting the residue calculated by the above analysis operation to the display unit 111 and/or a predetermined device. Further, the arithmetic unit 91 may store the fact that the analysis result of this time is normal in the storage unit 93.

In this way, it is possible to determine that analysis of the analysis target gas (measurement target component) has been performed in a normal condition, based on the above output.

If the analysis result is normal, the arithmetic unit 91 outputs the analysis result, e.g., causes the display unit 111 to display the analysis result of the analysis target gas (Step S43).

On the other hand, if the residue is a predetermined threshold value or more (“Yes” in Step S41), the arithmetic unit 91 outputs a signal for indicating that the analysis result is abnormal (Step S44). For instance, the arithmetic unit 91 outputs a signal for displaying that the analysis result is abnormal to the display unit 111, outputs a signal producing sound for informing that the analysis result is abnormal to a predetermined device, and/or outputs a signal for outputting that analysis result is abnormal to a predetermined device. In addition, the arithmetic unit 91 may output the signal for outputting the residue calculated in the above analysis operation, to the display unit 111 and/or predetermined device. Further, the arithmetic unit 91 may store the fact that the analysis result of this time is abnormal in the storage unit 93.

In this way, based on the output described above, it is possible to determine that analysis of the analysis target gas has not been performed in a normal condition (i.e., the analysis result is abnormal).

In this way, the analysis device 100 outputs the signal for determining whether or not the analysis result of the analysis target gas is abnormal, based on a difference between the detection signal information about the detection signal obtained by causing the sample gas SG to pass through the measurement light L (such as the sample correlation value), and the reference signal information about the reference signal indicating absorption of the measurement light L by the analysis target gas (and/or interference gas) (e.g., the single correlation value).

The residue between the detection signal information and the reference signal information indicates an amount of shift between the detection signal that is actually detected and the reference signal obtained in advance, and is calculated in predetermined information processing using the detection signal information and the reference signal information (e.g., in the process of solving the simultaneous equations of Equation 2).

As described above, the residue is calculated as shift amounts between a plurality of coordinate points (sᵢ₁, sᵢ₂, . . . , sᵢₘ, S_{di}) of the actually calculated sample correlation values and the single correlation values, and the curve expressed by S=s₁*C1+s₂*C2+ . . . sₘ*Cm calculated by regression analysis of the plurality of coordinate points (e.g., the sum of squares of distances between the above curve and the coordinate points).

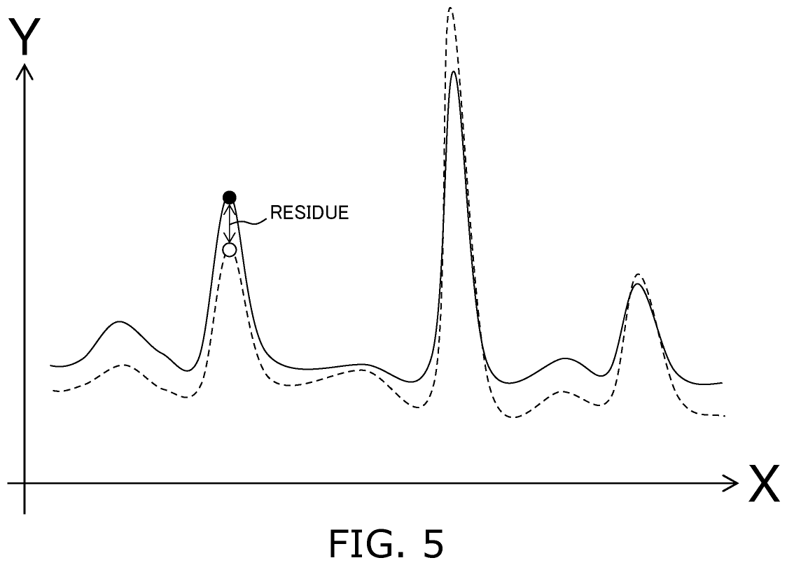

As illustrated in FIG. 5, the above residue corresponds to the residue between the measured data that is actually measured (illustrated by the solid line in FIG. 5) and reference data for analyzing the analysis target gas (illustrated by the broken line in FIG. 5) (the sum of squares of the differences between individual points of the measured data and corresponding points of the reference data). Note that in FIG. 5, if the measured data and the reference data are absorption spectra, the X axis represents wavelength, while the Y axis represents absorbance. In addition, if the measured data and the reference data are signals, the X axis represents time, while the Y axis represents detection signal. FIG. 5 is a diagram illustrating meaning of the residue.

In other words, if the residue is small, it means that the shift between the measured data and the reference data is small. In this case, the detection signal that is actually detected substantially corresponds to the reference signal obtained in advance (i.e., there is little dissociation between the detection signal and the reference signal), and it can be determined that normal analysis is performed using the detection signal that is actually detected by the detection unit 75.

On the other hand, if the residue is large, it means that the shift between the measured data and the reference data is large. In this case, the detection signal that is actually detected does not correspond to the reference signal obtained in advance (i.e., there is much dissociation between the detection signal and the reference signal), and it can be determined that normal analysis cannot be performed using the detection signal that is actually detected by the detection unit 75 (the analysis result is abnormal), or that an accurate analysis result is not obtained.

In addition, evaluation of the analysis result based on the residue is more advantageous than evaluation of the analysis result based on a shape of the detection signal due to the following point. For instance, there is a case where the analysis target gas can be appropriately analyzed, even if the detection signal contains noise so that the shape of the detection signal is changed from that in the normal case. In this case, in the evaluation based on the shape of the detection signal, it may be determined that the analysis is not appropriately performed. In contrast, in the evaluation based on the residue, the residue is calculated to be small for data in which the analysis result is appropriately performed, and hence it is highly possible that it is correctly determined that the analysis result is appropriate.

In addition, for example, there is a case where the analysis of the analysis target gas is not appropriate, even if the shape of the detection signal does not have abnormality at a glance. In this case, in the evaluation based on the shape of the detection signal, it may be determined that the analysis is appropriately performed. In contrast, in the evaluation based on the residue, the residue is calculated to be large for data in which the analysis result is not appropriately performed, and hence it is highly possible that it is correctly determined that the analysis result is not appropriate.

In this way, the analysis device 100 can correctly and easily determine whether or not the analysis result of the analysis target gas is abnormal, without using a method requiring proficient experience.

In addition, in the analysis device 100, the arithmetic unit 91 outputs a signal for indicating that an analysis result of the analysis target gas is abnormal, if the residue is a predetermined threshold value or more. In this way, if the actually obtained detection signal is largely dissociated from the reference signal, it can be appropriately determined that the analysis result of the analysis target gas is abnormal.

### 2. Second Embodiment

In the analysis device 100, an analysis result of the analysis target gas may be affected by various influences. These influences are roughly classified into influences of the analysis device 100 and influences of the sample gas SG. As the influences of the analysis device 100, there are, for example, temperature of the analysis device 100, pressure of the sample gas SG in the sampling space SS, wavelength shift of the measurement light L (a difference between a wavelength of the measurement light L when acquiring the reference signal and a wavelength of the measurement light L when acquiring the detection signal), optical axis shifts of the element lights L1 to L4 and/or the measurement light L, abnormality (such as malfunction) of the light sources 51a to 51d, and the like.

On the other hand, as the influences of the sample gas SG, there are, for example, presence of interference components and/or accompanying components (high concentration gas components) in the sample gas SG, presence of gas components that are not considered when creating Equation 2 (referred to as unknown components), temperature and pressure of the sample gas SG, and the like. If there is an accompanying component, there may occur a phenomenon in which an absorption spectrum width is increased so that the absorption peak is lowered (referred to as broadening), for example. If there is an interference component/unknown component (that is not considered), there may occur a phenomenon in which a peak of the absorption spectrum appears in an unexpected wavelength, and the spectrum shape becomes “distorted”, for example.

Therefore, in the second embodiment, the arithmetic unit 91 outputs a signal for determining whether or not an analysis result of the analysis target gas is abnormal, based on not only the residue described above in the first embodiment, but also at least one of pressure of the sample gas SG in the sampling space SS, temperature of the analysis device 100 when acquiring the detection signal, temperature of the sample gas SG, a peak position of the spectrum created from the detection signal, a spread width of the spectrum. Note that “temperature of the analysis device 100” is temperature around the analysis device 100 or inside temperature of the analysis device 100.

Specifically, the arithmetic unit 91 outputs a signal for determining whether or not an analysis result is abnormal, based on not only whether or not the residue is a predetermined threshold value or more, but also whether or not each of pressure of the sample gas SG in the sampling space SS, temperature of the analysis device 100, peak position shift of the spectrum, spread width of the spectrum is largely deviated from a set reference value.

As described above, by outputting a signal for determining whether or not an analysis result is abnormal, in consideration of influences of other factors in addition to the residue, abnormality caused by the above factors can be specified.

### 3. Third Embodiment

In addition, when analyzing the analysis target gas contained in the sample gas SG, it is preferred to eliminate influences of the analysis device 100 and influences of the sample gas SG, as much as possible to consider. In particular, it is desired to eliminate a factor that will largely affect the analysis result. In this way, for example, if the residue described in the first embodiment is a predetermined threshold value or more, the arithmetic unit 91 can output a signal for determining that the sample gas SG contains unknown component other than the analysis target gas and/or the interference component to be considered.

For instance, it is preferred to eliminate influences of wavelength shift of the measurement light L, gas pressure inside the sampling space SS, and temperature of the sample gas SG (the analysis device 100).

For instance, influence of the wavelength shift of the measurement light L can be eliminated as follows. First, the intensity related signal (reference signal) is acquired for each of a plurality of types of the measurement light L having various wavelengths (ranges), in accordance with the method described in the first embodiment. After that, from the intensity related signal acquired for each wavelength, the single correlation value at the wavelength is extracted in accordance with the method described in the first embodiment.

When analyzing the analysis target gas, wavelength of the measurement light L when the detection signal is acquired from ambient temperature or the like is predicted, and using the single correlation value and the sample correlation value for this wavelength, the simultaneous equations of Equation 2 are created, to solve the simultaneous equations. In this way, it is possible to obtain the analysis result considering the wavelength shift, and it is possible to determine whether or not the analysis result is abnormal considering the wavelength shift. In other words, it is possible to determine whether or not the analysis result is abnormal by excluding influence of the wavelength shift.

For instance, influences of pressure and/or temperature of the gas inside the sampling space SS can be eliminated as follows. First, the measurement light L is passed through the sampling space SS filled with span gas at various pressures and/or various temperatures, and the intensity related signal is acquired for each of the various pressures (a plurality of pressures), in accordance with the method described in the first embodiment. After that, from the intensity related signal acquired at each pressure and/or temperature, the single correlation value at the pressure and/or temperature is extracted, in accordance with the method described in the first embodiment.

When analyzing the analysis target gas, pressure and/or temperature of the sampling space SS filled with the sample gas SG are measured, and the simultaneous equations of Equation 2 are created using the single correlation value and the sample correlation value corresponding to the measured pressure and/or temperature of the sampling space SS, to solve the simultaneous equations. In this way, it is possible to obtain the analysis result considering pressure and/or temperature of the sampling space SS, and it is possible to determine whether or not the analysis result is abnormal considering the pressure and/or temperature. In other words, it is possible to determine whether or not the analysis result is abnormal by excluding influences of pressure and/or temperature inside the sampling space SS.

### 4. Fourth Embodiment

In the embodiment described above, a signal for determining whether or not an analysis result is abnormal is output, based on the residue calculated by solving the simultaneous equations, which uses the detection feature quantity extracted from the detection signal, and the reference feature quantity extracted from the reference signal. However, this is not a limitation. It may be possible to output a signal for determining whether or not an analysis result is abnormal, based on the residue obtained by curve fitting between the absorption spectrum by the sample gas SG created from the detection signal (referred to as the detection absorption spectrum), and the absorption spectrum created from the reference signal obtained in advance by causing the analysis target gas and/or the interference gas to absorb the measurement light L (referred to as the reference absorption spectrum). In other words, the detection signal information may be the detection absorption spectrum, while the reference signal information may be the reference absorption spectrum. In addition, the reference absorption spectrum may be one that is created based on infrared absorption spectrum data or the like.

### 5. Other Embodiments

Although a plurality of embodiments of the present invention are described above, the present invention is not limited to the embodiments and can be variously modified within the scope of the invention without deviating from the spirit thereof. In particular, the plurality of embodiments and variations described in the specification can be arbitrarily combined as necessary.
(A) In the analysis operation performed in accordance with the flowcharts illustrated in FIGS. 2 to 4, the process details of each step and/or the process order of the steps can be appropriately modified within the scope of the invention without deviating from the spirit thereof.
(B) The determination whether or not an analysis result is abnormal, based on the residue between the detection signal information and the reference signal information described above, can be applied also to a device other than the analysis device 100 illustrated in FIG. 1, which analyzes a gas contained in the sample gas SG based on the measurement light after passing through the sample gas SG.

### 6. Features of Embodiments

The above embodiments can be described also as follows.
(1) An analysis device (e.g., the analysis device 100) includes an irradiation unit (e.g., the irradiation unit 5), a detection unit (e.g., the detection unit 75), and an arithmetic unit (e.g., the arithmetic unit 91). The irradiation unit includes a laser that emits measurement light (e.g., the measurement light L) toward a sample gas (e.g., the sample gas SG). The detection unit detects the measurement light after passing through the sample gas. The arithmetic unit analyzes a predetermined gas based on a detection signal obtained when the detection unit detects the measurement light. In addition, the arithmetic unit outputs a signal for determining whether or not the analysis result of the predetermined gas is abnormal, based on a residue between detection signal information based on the detection signal, and reference signal information based on a reference signal indicating absorption of the measurement light by the predetermined gas.

In the analysis device described above, based on the residue between detection signal information based on an actual detection signal obtained by causing the measurement light to pass through the sample gas, and reference signal information based on a reference signal indicating absorption of the measurement light by the predetermined gas, the signal for determining whether or not the analysis result of the predetermined gas is abnormal is output. The residue between the detection signal information and the reference signal information indicates an amount of shift between the detection signal that is actually detected and the reference signal, and is calculated by predetermined information processing using the detection signal information and the reference signal information. For this reason, in the analysis device described above, it can be easily determined whether or not the analysis result of the predetermined gas is abnormal, without using a method requiring proficient experience.
(2) In the analysis device (1) described above, the arithmetic unit may output a signal for indicating that the analysis result of the predetermined gas is abnormal, if the residue is a predetermined threshold value or more. In this way, if the detection signal actually obtained is largely dissociated from the reference signal obtained in advance, it can be appropriately determined that the analysis result of the predetermined gas is abnormal.
(3) In the analysis device (2) described above, the arithmetic unit may output a signal for indicating that the sample gas contains unknown gas other than the predetermined gas, if the residue is a predetermined threshold value or more. In this way, it is possible to know that the analysis result of the predetermined gas is abnormal because unknown gas is contained.
(4) In the analysis device (1) to (3) described above, the arithmetic unit may calculate the residue by regression analysis using the detection signal information and the reference signal information. In this way, a shift between the detection signal information and the reference signal information can be easily calculated.
(5) In the analysis device (1) to (4) described above, the detection signal information may include detection feature quantity that indicates features of the detection signal. In addition, the reference signal information may include reference feature quantity that indicates features of the reference signal. In this way, the number of parameters to be used for calculating the residue can be reduced.
(6) In the analysis device (1) to (4) described above, the detection signal information may be a detection absorption spectrum generated based on the detection signal. The reference signal information may be a reference absorption spectrum generated based on the reference signal. In this way, for example, the residue can be calculated based on comparison between the detection absorption spectrum and the reference absorption spectrum, for example, and it can be determined whether or not the analysis result is abnormal.
(7) In the analysis device (1) to (6) described above, the arithmetic unit may output a signal for determining whether or not the analysis result of the predetermined gas is abnormal, based on not only the residue but also at least one of sample gas pressure, temperature of the analysis device, a peak position of a spectrum generated from the detection signal, and a spread width of the spectrum, when the detection signal is obtained. In this way, it is possible to identify abnormality caused by sample gas pressure, temperature of the analysis device, or a peak position of the spectrum generated from the detection signal.
(8) An analysis method includes:
causing a laser to emit measurement light toward a sample gas (e.g., Step S1);
detecting the measurement light after passing through the sample gas (e.g., Step S2);
analyzing the predetermined gas based on a detection signal obtained by detecting the measurement light (e.g., Step S3); and
determining whether or not the analysis result of the predetermined gas is abnormal, based on a residue between detection signal information based on the detection signal, and reference signal information based on a reference signal indicating absorption of the measurement light by the predetermined gas (e.g., Step S4).

In the analysis method described above, based on the residue between detection signal information based on an actual detection signal obtained by causing the measurement light to pass through the sample gas, and reference signal information based on a reference signal indicating absorption of the measurement light by the predetermined gas, a signal for determining whether or not the analysis result of the predetermined gas is abnormal is output. The residue between the detection signal information and the reference signal information indicates an amount of shift between the detection signal that is actually detected and the reference signal, and is calculated by predetermined information processing using the detection signal information and the reference signal information. For this reason, in the analysis method described above, it can be easily determined whether or not the analysis result of the predetermined gas is abnormal, without using a method requiring proficient experience.

### INDUSTRIAL APPLICABILITY

The present invention can be widely applied to analysis devices that analyze a predetermined gas contained in a sample gas.

### REFERENCE SIGNS LIST

100 analysis device
1 casing
11 discharge port
3 filling unit
31 inlet
32 outlet
33a first reflection member
33b second reflection member
SS sampling space
5 irradiation unit
51a to 51d light source
L1 to L4 element light
L measurement light
7 propagation unit
71a first mirror
71b second mirror
71c third mirror
71d fourth mirror
71e fifth mirror
71f sixth mirror
73a first optical element
73b second optical element
73c third optical element
75 detection unit
TS propagation space
9 control unit
91 arithmetic unit
93 storage unit
111 display unit
W optical window
SG sample gas

1. An analysis device configured to analyze a predetermined gas contained in a sample gas, the device comprising:
an irradiation unit including a laser configured to emit measurement light toward the sample gas;
a detection unit configured to detect the measurement light after passing through the sample gas;
an arithmetic unit configured to analyze the predetermined gas based on a detection signal obtained when the detection unit detects the measurement light, wherein
the arithmetic unit outputs a signal for determining whether or not the analysis result of the predetermined gas is abnormal, based on a residue between detection signal information based on the detection signal, and reference signal information based on a reference signal indicating absorption of the measurement light by the predetermined gas.

2. The analysis device according to claim 1, wherein the arithmetic unit outputs a signal for indicating that the analysis result of the predetermined gas is abnormal, if the residue is a predetermined threshold value or more.

3. The analysis device according to claim 2, wherein the arithmetic unit outputs a signal for indicating that the sample gas contains unknown gas other than the predetermined gas, if the residue is a predetermined threshold value or more.

4. The analysis device according to claim 1, wherein the arithmetic unit calculates the residue by regression analysis using the detection signal information and the reference signal information.

5. The analysis device according to claim 1, wherein
the detection signal information includes detection feature quantity that indicates features of the detection signal, and
the reference signal information includes reference feature quantity that indicates features of the reference signal.

6. The analysis device according to claim 1, wherein the detection signal information is a detection absorption spectrum generated based on the detection signal, and the reference signal information is a reference absorption spectrum generated based on the reference signal.

7. The analysis device according to claim 1, wherein the arithmetic unit outputs a signal for determining whether or not the analysis result of the predetermined gas is abnormal, based on not only the residue but also at least one of sample gas pressure, temperature of the analysis device, a peak position of a spectrum generated from the detection signal, and a spread width of the spectrum, when the detection signal is obtained.

8. An analysis method of analyzing a predetermined gas contained in a sample gas, the method comprising:
causing a laser to emit measurement light toward a sample gas;
detecting the measurement light after passing through the sample gas;
analyzing the predetermined gas based on a detection signal obtained by detecting the measurement light; and
outputting a signal for determining whether or not the analysis result of the predetermined gas is abnormal, based on a residue between detection signal information based on the detection signal, and reference signal information based on a reference signal indicating absorption of the measurement light by the predetermined gas.

9. A non-transitory computer readable medium storing a program causing a computer to perform an analysis method of analyzing a predetermined gas contained in a sample gas, the analysis method comprising:
causing a laser to emit measurement light toward a sample gas;
detecting the measurement light after passing through the sample gas;
analyzing the predetermined gas based on a detection signal obtained by detecting the measurement light; and
outputting a signal for determining whether or not the analysis result of the predetermined gas is abnormal, based on a residue between detection signal information based on the detection signal, and reference signal information based on a reference signal indicating absorption of the measurement light by the predetermined gas.
